(19) United States
(12) Patent Application Publication
(10) Pub. No.: US 2018/0319774 A1
(43) Pub. Date: Nov. 8, 2018

(54) IRIDIUM (III) COMPLEXES WITH CYCLIC QUINOXALINE-FUSED LIGANDS AND ORGANIC LIGHT-EMITTING DIODES USING THE SAME

(71) Applicant: JUST ABOUT SHOWING CO., LTD., New Taipei City (TW)

(72) Inventor: Jen-Cherng LEE, New Taipei City (TW)

(21) Appl. No.: 15/584,564

(22) Filed: May 2, 2017

(51) Int. Cl. C07D 403/02; C07D 241/36; C07D 521/00; C09K 11/06; C07D 403/04

(52) U.S. Cl. CPC C07D 403/02; C07D 241/36; C07D 403/04; C09K 11/06; C07D 521/00

(57) ABSTRACT

An iridium (III) complex compound with cyclic quinoxaline-fused dibenzosuberene ligand is shown in General Formula (1),

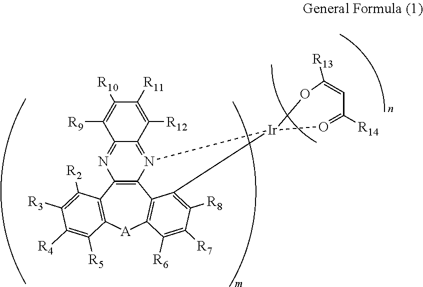

wherein m is 1 or 2, n is 1 or 2, and m+n is 3; A is a bridging atom represented by General Formula (2) or General Formula (3),

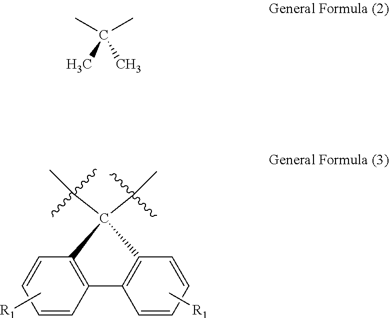

wherein R₁ is a hydrogen atom, alkyl or tert-butyl group, R₂ to R₁₄ are independently selected from the group consisting of hydrogen atom, halogen atom, cyano group, alkyl group, cycloalkyl group, aryl group, alkoxy group, amino group, thioalkyl group, silyl group and alkenyl group.

### BACKGROUND OF THE INVENTION

### Field of Invention

The present invention relates to an organic electroluminescent material and an organic electroluminescent device using the same and, in particular, to a series of iridium (III) complex compounds with cyclic quinoxaline-fused cis-stilbene (dibenzosuberene) ligands and the organic electroluminescent device using the same.

### Related Art

With the advances in electronic technology, a light weight and high efficiency flat display device has been developed. An organic electroluminescent device becomes the mainstream of the next generation flat panel display device due to its advantages of self-luminosity, no restriction on viewing angle, power conservation, simple manufacturing process, low cost, high response speed, full color and so on.

In general, the organic electroluminescent device includes an anode, an organic luminescent layer and a cathode. When applying a direct current to the organic electroluminescent device, holes and electrons are injected into the organic luminescent layer from the anode and the cathode, respectively. Charge carriers move and then recombine in the organic luminescent layer because of the potential difference caused by an applied electric field. The excitons generated by the recombination of the electrons and the electron holes may excite the luminescent molecules in the organic luminescent layer. The excited luminescent molecules then release the energy in the form of light.

Nowadays, the organic electroluminescent device usually adopts a host-guest emitter system. The organic luminescent layer disclosed therein includes a host material and a guest material. The holes and the electrons are transmitted to the host material and then further transferred to the guest material to form excitons and then generate light. The guest material can be categorized into fluorescent material and phosphorescent material. Theoretically, the internal quantum efficiency can approach 100% by using appropriate phosphorescent materials. Therefore, the phosphorescent materials recently have become one of the most important developments in the field of organic electroluminescent materials. Among the currently commercially available organic electroluminescent materials, the most commonly used phosphorescent materials are the iridium (VIII B)-based metal complex compounds, including, for example, Tris[2-phenylpyridinato-C2,N]iridium(III) (Ir(ppy)3) of a green phosphorescent material and Bis[2-(4,6-difluorophenyl)pyridinato-C2,N](picolinato)iridium(III) (FIrpic) of a blue phosphorescent material.

Moreover, the selection of organic electroluminescent material is not only based on the matching of HOMO and LUMO energy levels but also counts on the high decomposition temperature in order to avoid pyrolysis during thermal vacuum deposition and also thus avoid the decrease in thermal stability.

Accordingly, it is an urgent need to provide an organic electroluminescent material and an organic electroluminescent device using the same which have high luminous efficiency and excellent thermal stability.

### SUMMARY OF THE INVENTION

In view of the foregoing objectives, the disclosure provides a series of iridium (III) complex compounds with cyclic quinoxaline-fused dibenzosuberene ligands to be used as organic electroluminescent materials. The present disclosure also provides an organic electroluminescent device using the same. The iridium (III) complex compounds with cyclic quinoxaline-fused dibenzosuberene ligands have excellent optical and optoelectronic effects and thermal stability.

An iridium (III) complex compound with cyclic quinoxaline-fused dibenzosuberene ligand according to the present disclosure has a structure of the following General Formula (1).

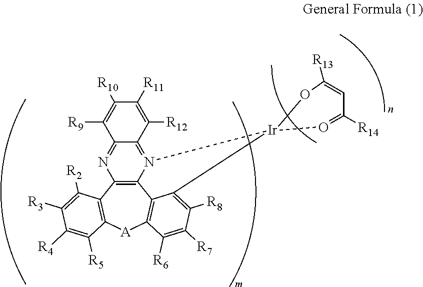

In the General formula (1), m is 1 or 2, n is 1 or 2, and m+n is 3. A is a bridging atom represented by General Formula (2) or General Formula (3).

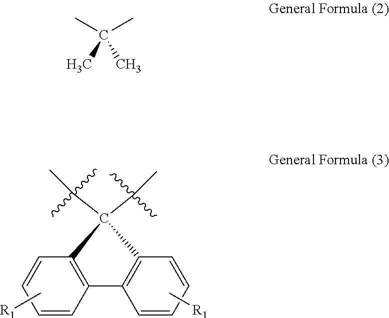

Herein, R₁ is a hydrogen atom, alkyl, or tert-butyl group. R₂ to R₁₄ are independently selected from the group consisting of hydrogen atom, halogen atom, cyano group, alkyl group, cycloalkyl group, aryl group, alkoxy group, amino group, haloalkyl group, thioalkyl group, silyl group and alkenyl group.

In one embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6. The cycloalkyl group is a substituted or unsubstituted cycloalkyl group with the carbon number of 3 to 6. The alkoxy group is selected from the group consisting of a substituted or unsubstituted straight-chain alkoxy group with the carbon number of 1 to 6, and a substituted or unsubstituted branched-chain alkoxy group with the carbon number of 3 to 6. The amino group is selected from the group consisting of secondary amino group and tertiary amino group. The haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6. The thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6. The silyl group is selected from the group consisting of a substituted or unsubstituted straight-chain silyl group with the carbon number of 1 to 6, and a substituted or unsubstituted branched-chain silyl group with the carbon number of 3 to 6. The alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

In one embodiment, the iridium (III) complex compound with cyclic quinoxaline-fused dibenzosuberene ligand can be represented by the General Formula (1) and m is 2 and n is 1.

In one embodiment, the iridium (III) complex compound with cyclic quinoxaline-fused dibenzosuberene ligand can be represented by the following chemical formula 1 or 2.

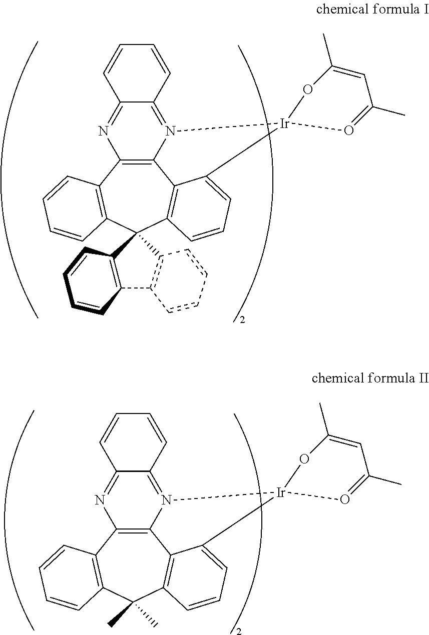

In one embodiment, the iridium (III) complex compounds with cyclic quinoxaline-fused dibenzosuberene ligands have decomposition temperatures (T_{d}) ranged from 361° C. to 371° C.

In one embodiment, the iridium (III) complex compounds with cyclic quinoxaline-fused dibenzosuberene ligands have oxidation potentials ranged from 0.55V to 0.60V and redox potentials ranged from −1.75V to −2.13V.

In one embodiment, the iridium (III) complex compounds with cyclic quinoxaline-fused dibenzosuberene ligands have highest occupied molecular orbital energy level (E_{HOMO}) ranged from −5.35 eV to −5.4 eV and lowest unoccupied molecular orbital energy level (E_{LUMO}) ranged from −3.0 eV to −3.1 eV.

An organic electroluminescent device which is also provided includes a first electrode layer, a second electrode layer and an organic luminescent unit. The organic luminescent unit is deposited between the first electrode layer and the second electrode layer. The organic luminescent unit has at least an iridium (III) complex compound with cyclic quinoxaline-fused dibenzosuberene ligand as shown in General Formula (1).

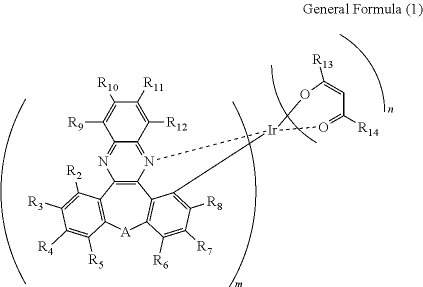

In the General formula (1), m is 1 or 2, n is 1 or 2, and m+n is 3. A is a bridging atom represented by General Formula (2) or General Formula (3).

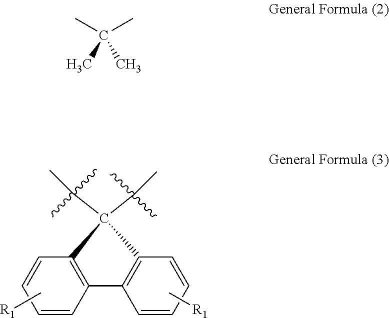

Herein, R₁ is a hydrogen atom, alkyl or tert-butyl group, R₂ to R₁₄ are independently selected from the group consisting of hydrogen atom, halogen atom, cyano group, alkyl group, cycloalkyl group, aryl group, alkoxy group, amino group, thioalkyl group, silyl group and alkenyl group.

In one embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

In one embodiment, the iridium (III) complex compound with cyclic quinoxaline-fused dibenzosuberene ligand can be represented by the General Formula (1) and m is 2 and n is 1.

In one embodiment, the iridium (III) complex compound with cyclic quinoxaline-fused dibenzosuberene ligand can be represented by the following chemical formula 1 or 2.

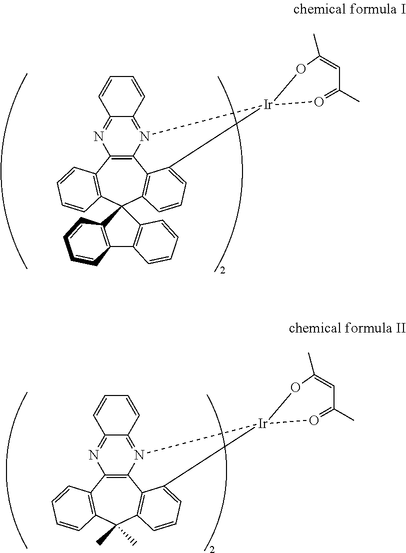

In one embodiment, the organic luminescent unit comprises an organic luminescent layer.

In one embodiment, the organic luminescent unit further comprises a hole transport layer and an electron transport layer, and the organic luminescent layer is deposited between the hole transport layer and the electron transport layer.

In one embodiment, the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

In one embodiment, the organic luminescent layer comprises a host material and a guest material, and the guests material comprises the iridium (III) complex compound with cyclic quinoxaline-fused dibenzosuberene ligand.

In one embodiment, the content of the guest material in the organic luminescent layer is between 3 wt % to 20 wt %.

As mentioned above, in the iridium (III) complex compound with cyclic quinoxaline-fused dibenzosuberene ligand according to the present disclosure, the cyclic ligand (i.e., di-methyl-dibenzosuberene[d]quinoxaline or spirofluorene-dibenzosuberene[d]quinoxaline) is sterically hindered by its quinoxaline-fused dibenzosuberene and/or dimethyl or spirofluorene fragment so that the molecule has a non-coplanar structure. Such molecular configuration may reduce the concentration quenching effect caused by intermolecular stacking. Therefore, the series of the iridium (III) complex compounds according to the present disclosure are suitable for a guest organinc luminescent material of a phosphorescent organic electroluminescent device with excellent thermal stability and high luminous efficiency.

### BRIEF DESCRIPTION OF THE DRAWINGS

The embodiments will become fully understood from the detailed description and accompanying drawings, which are given for illustration only, and thus are not limitative of the present invention, and wherein:

FIG. 1 is a cross-sectional schematic diagram of an organic electroluminescent device of the second embodiment according to the invention;

FIG. 2 is a cross-sectional schematic diagram of an organic electroluminescent device of the third embodiment according to the invention;

FIG. 3 is a cross-sectional schematic diagram of an organic electroluminescent device of the fourth embodiment according to the invention;

FIG. 4 depicts the chemical structure of the compound of Chemical Formula (1): Ir(QSTIF)₂(acac) according to one embodiment of the invention; and

FIG. 5 depicts the chemical structure of the compound of Chemical Formula (2): Ir(DMQSTI)₂(acac) according to one embodiment of the invention.

### DETAILED DESCRIPTION OF THE INVENTION

The embodiments of the invention will be apparent from the following detailed description, which proceeds with reference to the accompanying drawings, wherein the same references relate to the same elements.

Iridium (III) Complex Compound with Cyclic Quinoxaline-Fused Dibenzosuberene Ligand

An iridium (III) complex compound with cyclic quinoxaline-fused dibenzosuberene ligand according to the first embodiment of the present invention has a structure of the following General Formula (1).

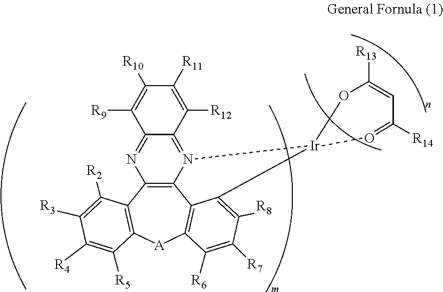

In General formula (1), m is 1 or 2, n is 1 or 2, and m+n is 3. A is a bridging atom represented by General Formula (2) or General Formula (3).

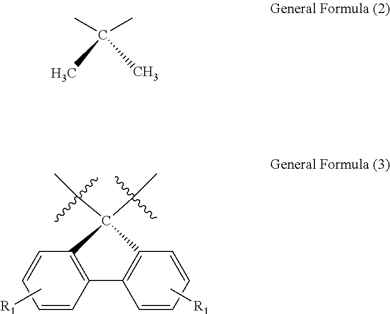

In General Formula (3), R₁ is a hydrogen atom, alkyl or tert-butyl group. In General Formula (1), R₂ to R₁₄ are independently selected from the group consisting of hydrogen atom, halogen atom, cyano group, alkyl group, cycloalkyl group, aryl group, alkoxy group, amino group, haloalkyl group, thioalkyl group, silyl group and alkenyl group.

In the present embodiment, the alkyl group is selected from the group consisting of a substituted or unsubstituted straight-chain alkyl group with the carbon number of 1 to 6, and a substituted or unsubstituted branched-chain alkyl group with the carbon number of 3 to 6. The cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6. Moreover, the secondary amino group can be an amino group having one aromatic ring substitutent (for example, a phenyl amino group) or having one C₁-C₆ radical of straight-chain alkyl, branch-chain alkyl, or non-aromatic ring (for example, a methyl amino group). The tertiary amino group can be an amino group having two independent aromatic ring substitutents (for example, a diphenyl amino group, —NPh₂) or having two independent C₁-C₆ radicals of straight-chain alkyl, branch-chain alkyl, or non-aromatic ring (for example, a dimethyl amino group). The aryl group may be an aromatic hydrocarbon with the carbon number of 6 to 16 or a hetero aromatic ring with the carbon number of 5 to 16.

The iridium (III) complex compound with cyclic quinoxaline-fused dibenzosuberene ligand according to the present embodiment represented by General Formula (1) can be a guest material of an organic luminescent layer in an organic electroluminescent device. An example is the compound of General Formula (1-1), where m=2 and n=1 in General Formula (1).

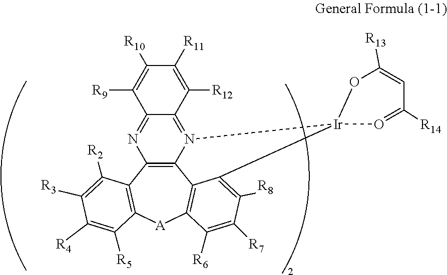

When the bridging atom A in General Formula (1) is represented by General formula (3), the iridium (III) complex compound with cyclic quinoxaline-fused dibenzosuberene ligand according to the present embodiment is then represented by the following General Formula (1-2).

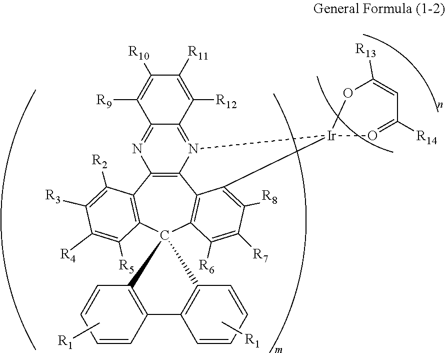

On the other hand, when the bridging atom A in General Formula (1) is represented by General formula (2), the iridium (III) complex compound with cyclic quinoxaline-fused dibenzosuberene ligand according to the present embodiment is then represented by the following General Formula (1-3).

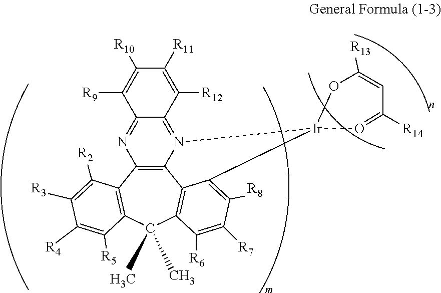

An example of the present embodiment is the compound of Chemical Formula (1), Ir(QSTIF)₂(acac), where the bridging atom A is represented by General Formula (3), R₁ to R₁₂ are all hydrogen atoms and R₁₃ to R₁₄ are both methyl groups in General Formula (1-1).

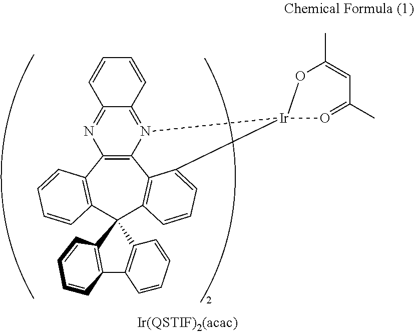

Alternatively, another example of the present embodiment is the compound of Chemical Formula (2), Ir(DMQSTI)₂(acac), where the bridging atom A is represented by General Formula (2), R₁ to R₁₂ are all hydrogen atoms and R₁₃ to R₁₄ are both methyl groups in General Formula (1-1).

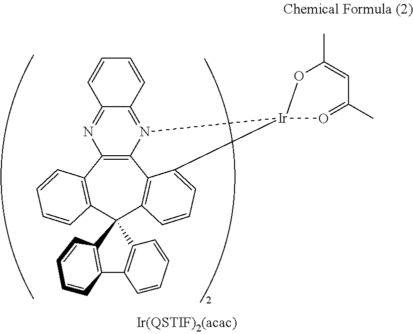

In the iridium (III) complex compound with cyclic quinoxaline-fused dibenzosuberene ligand according to the present embodiment, the cyclic ligand (e.g., the ligand of the compound of General Formula (1-3), di-methyl-dibenzosuberene[d]quinoxaline, or the ligand of the compound of General Formula (1-2), spirofluorene-dibenzosuberene[d]quinoxaline) is sterically hindered by its quinoxaline-fused dibenzosuberene and/or dimethyl or spirofluorene fragment so that the molecule has a non-planar structure. Such molecular configuration may reduce the concentration quenching effect caused by intermolecular stacking. Therefore, the series of the iridium (III) complex compounds according to the present embodiment are suitable for a guest organinc luminescent material of a phosphorescent organic electroluminescent device with excellent thermal stability and high luminous efficiency.

In the present embodiment, the iridium (III) complex compounds with cyclic quinoxaline-fused dibenzosuberene ligands have decomposition temperatures (T_{d}) ranged from 361° C. to 371° C. and have oxidation potentials ranged from 0.55V to 0.60V and redox potentials ranged from −1.75V to −2.13V. In addition, the highest occupied molecular orbital energy levels (E_{HOMO}) of the the iridium (III) complex compounds are ranged from −5.35 eV to −5.4 eV and their lowest unoccupied molecular orbital energy levels (E_{LUMO}) are ranged from −3.0 eV to −3.1 eV.

The thermal, optical, and electrochemical properties of the series of the iridium (III) complex compounds with cyclic quinoxaline-fused dibenzosuberene ligands according to the present embodiment are further illustrated in the following experimental examples.

Organic Electroluminescent Device

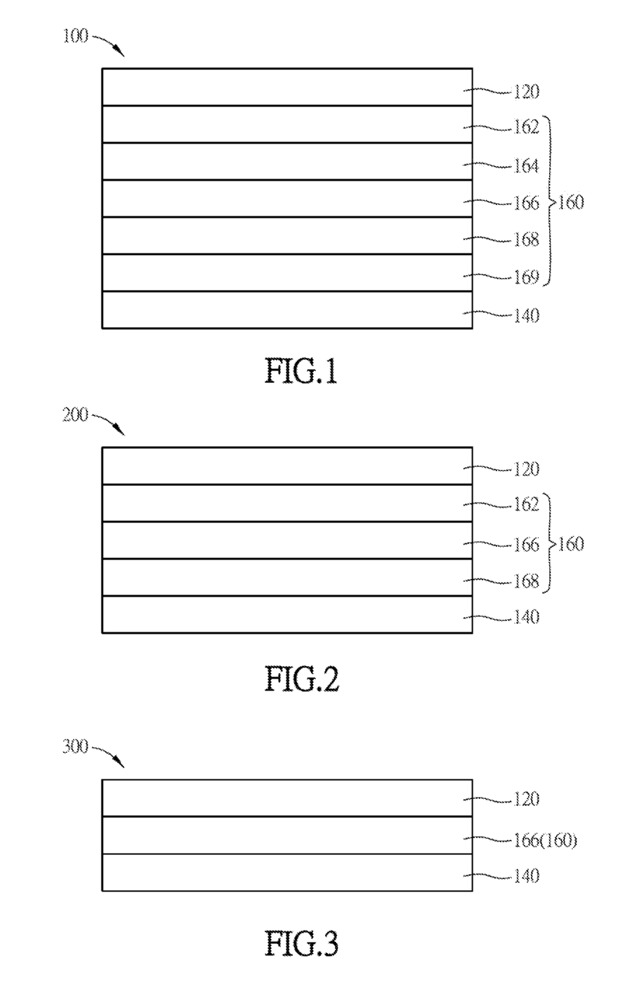

Please refer to FIG. 1, an organic electroluminescent device 100 of the second embodiment according to the disclosure includes a first electrode layer 120, a second electrode layer 140 and an organic luminescent unit 160. In the embodiment, the first electrode layer 120 can be a transparent electrode material, such as indium tin oxide (ITO), and the second electrode layer 140 can be a metal, transparent conductive substance or any other suitable conductive material. On the other hand, the first electrode layer 120 can also be a metal, transparent conductive substance or any other suitable conductive material, and the second electrode layer 140 can also be a transparent electrode material. Overall, at least one of the first electrode layer 120 and the second electrode layer 140 of the embodiment is a transparent electrode material, so that the light emitted from the organic luminescent unit 160 may pass through the transparent electrode, thereby enabling the organic electroluminescent device 100 to emit light.

In addition, please also refer to FIG. 1, the organic luminescent unit 160 can comprise a hole injection layer 162, an hole transport layer 164, an organic luminescent layer 166, an electron transport layer 168 and an electron injection layer 169. The hole transport layer 164, the organic luminescent layer 166 and the electron transport layer 168 are sequentially deposited between the hole injection layer 162 and the electron injection layer 169.

Herein, the materials of the hole injection layer 162 may be poly(3,4-ethylenedioxythiophene) polystyrene sulfonate (PEDOT:PSS) or Hexaazatriphenylenehexacarbonitrile (HATCN). Moreover, the thickness of the hole injection layer 162 of the embodiment is, for example, greater than 0 nm and no more than 50 nm. The materials of the hole transport layer 164 can be 1,1-Bis[4-[N,N′-di(p-tolyl)amino]phenyl]cyclohexane (TAPC), Tris(4-carbazoyl-9-ylphenyl)amine (TCTA), N,N-bis-(1-naphthyl)-N,N-diphenyl-1,1-biphenyl-4,4-diamine (NPB), or N—N′-diphenyl-N—N′bis(3-methylphenyl)-[1-1′-biphenyl]-4-4′-diamine (TPD) and so on. In the embodiment, the hole injection layer 162 and the hole transport layer 164 can increase the injection rate of hole transport from the first electrode layer 120 to the organic luminescent layer 166 and can also reduce the driving voltage of the organic electroluminescent device 100.

The materials of the electron transport layer 168 may be 2,2,2-(1,3,5-benzinetriyl)-tris(1-phenyl-1-H-benzimidazole) (TBPI), 1,3,5-Tri(m-pyridin-3-ylphenyl)benzene (TmPyPb), or 1,3-Bis[3,5-di(pyridin-3-yl)phenyl]benzene (B3PyPb). In the embodiment, the thickness of the electron transport layer 168 is, for example, greater than 0 nm and no more than 50 nm. The the electron transport layer 168 may further increase the transport rate of the electron from the electron injection layer 169 to the organic luminescent layer 166.

In addition, the thickness of the organic luminescent layer 166 of the embodiment is between 5 nm and 30 nm, for example, 15 nm or 25 nm. The organic luminescent layer 166 includes the host material and the guest material, and the guest material can be the above-mentioned organic electroluminescent material which has a structure of General Formula (1). The doping concentration of the guest materials can be between 3 wt % and 20 wt %, for example, 3, 5, 7, 10, 15 or 20 wt %. The host materials can be 4,4′-Bis(N-carbazolyl)-1,1′-biphenyl (CBP), N,N′-Di(1-naphthyl)-N,N′-diphenyl-(1,1′-biphenyl)-4,4′-diamine (NBP), 3,5-di(9H-carbazol-9-yl)tetraphenylsilane (SimCP2), 4,4′,4″-tri(N-carbazolyl)triphenylamine (TCTA), 2,7-bis(carbazo-9-yl)-9,9-ditolyfluorene (Spiro-2CBP), or Bis(10-hydroxybenzo[h]quinolinato)beryllium (Bebq₂).

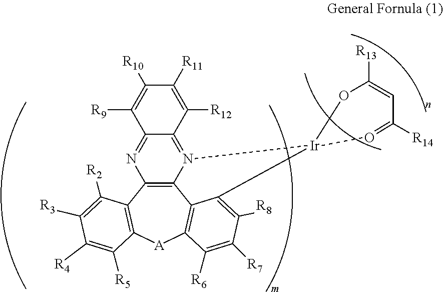

In General formula (1), m is 1 or 2, n is 1 or 2, and m+n is 3. A is a bridging atom represented by General Formula (2) or General Formula (3).

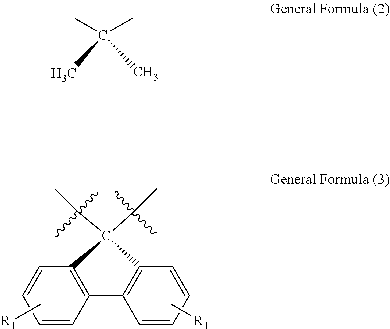

In General Formula (3), R₁ is a hydrogen atom, alkyl or tert-butyl group. In General Formula (1), R₂ to R₁₄ are independently selected from the group consisting of hydrogen atom, halogen atom, cyano group, alkyl group, cycloalkyl group, aryl group, alkoxy group, amino group, haloalkyl group, thioalkyl group, silyl group and alkenyl group.

In addition, the various examples and the selection of the substituents of R₂ to R₁₄ of General Formula (1), as well as their properties, such as their decomposition temperatures (T_{d}), oxidation potentials, redox potentials, highest occupied molecular orbital energy levels (E_{HOMO}), and lowest unoccupied molecular orbital energy levels (E_{LUMO}), are substantially the same as those in the first embodiment and are therefore omitted here.

In addition, FIG. 2 is a cross-sectional schematic diagram of an organic electroluminescent device 200 of the third embodiment according to the invention. The configuration of the organic electroluminescent device 200 is substantially similar with that of the organic electroluminescent device 100, and same elements have substantial the same characteristics and functions. Therefore, the similar references relate to the similar elements, and detailed explanation is omitted hereinafter.

Please refer to FIG. 2, in the embodiment, the organic luminescent unit 160 can comprise a hole injection layer 162, an organic luminescent layer 166 and an electron transport layer 168. The organic luminescent layer 166 is deposited between the hole injection layer 162 and the electron transport layer 168.

In addition, FIG. 3 is a cross-sectional schematic diagram of an organic electroluminescent device 300 of the fourth embodiment according to the invention. The configuration of the organic electroluminescent device 300 is substantially similar with that of the organic electroluminescent device 100, and same elements have substantial the same characteristics and functions. Therefore, the similar references relate to the similar elements, and detailed explanation is omitted hereinafter.

Please refer to FIG. 3, in the embodiment, the organic luminescent unit 160 can comprise an organic luminescent layer 166.

The configuration of the organic electroluminescent device according to the invention is not limited to what is disclosed in the second, third or fourth embodiment. The second, third and fourth embodiments are embodiments for illustration.

To illustrate the synthesis the series of the iridium (III) complex compounds with cyclic quinoxaline-fused dibenzosuberene ligands according to the embodiment, there are several examples shown below.

### Example 1: Synthesis of Compound 9 (Spiro-Fluorene-Dibenzosuberene)

A stir bar was placed in a 500 ml tri-neck flask. 5H-Dibenzosuberenone (8.25 g, 40 mmol) were first added to a addition funnel which is installed on the tri-neck flask. 2-bromobiphenyl (10.4 ml, 60 mmol) and tetrahydrofuran (180 ml) were sequencially added to the tri-neck flask after the flask was dried by vacuum, followed by injecting slowly 1.6 M n-butyllithium hexane solution (37.5 ml, 60 mmol) into the tri-neck flask at −78° C. After reaction for 30 minutes, 140 ml of tetrahydrofuran was added to the addition funnel, and then the stop-valve was opened such that the 5H-Dibenzosuberenone solution was added to the reaction solution in the flask. After reaction for 6 hours, saturated sodium bicarbonate solution was added to quench the reaction. The solvent was removed by rotator evaporation and the reaction mixture was then extracted by dichloromethane (3×100 ml). The combined extractant was then dried by adding anhydrous magnesium sulfate, followed by filtration and condensation with a rotary evaporator to obtain the intermediate product.

Another stir bar was placed into a 250 ml single-neck round-bottom flask. The intermediate product obtained above was added to the round-bottom flask, followed by sequencially adding 100 ml of acetic acid and 2 ml of concentrated hydrogen chloride. The color of the resulting reaction solution turned deep purple. The round-bottom flask with the reaction mixture was then equipped to a reflux system and the reaction mixture was refluxed for 12 hours. The round-bottom flask was then removed from the reflux system and cooled down to room temperature. n-hexane (150 ml) was added to the flask on an ice bath. After stirring, solid precipitates were collected by a filtering funnel and followed by washing with hexane for three times. After drying, compound 9 as a white solid (12.88 g, yield: 94%) was obtained. The above reaction is represented by the chemical equation (1-1).

Spectral data as follow: Tₘ 237° C. (DSC); M.W.: 342.43; ¹H NMR (400 MHZ, CDCl₃) δ 7.97 (d, J=7.8, 2H), 7.73 (d, J=7.5, 2H), 7.38 (dd, J=8.0, 1.1, 2H), 7.35 (td, J=4.3, 1.5, 2H), 7.24 (td, J=7.5, 1.1, 2H), 7.19 (td, J=7.1, 1.3, 2H), 6.96 (s, 2H), 6.95-6.87 (m, 4H); ¹³C NMR (100 MHz, CDCl₃) δ 153.0, 141.8, 138.9, 136.4, 133.3, 132.2, 128.8, 128.4, 127.9, 127.5, 127.2, 127.1, 120.2, 66.0; MS (FAB) 342.1 (M+, 100); TLC R_{f} 0.5 (CH₂Cl₂/hexane, 1/3); HR-MS Anal. calcd for C₂₇H₁₈: 342.1403, found: 342.1401; Anal. Calcd for C₂₇H₁₈: C, 94.70, H, 5.30. Found: C, 94.65, H, 5.31.

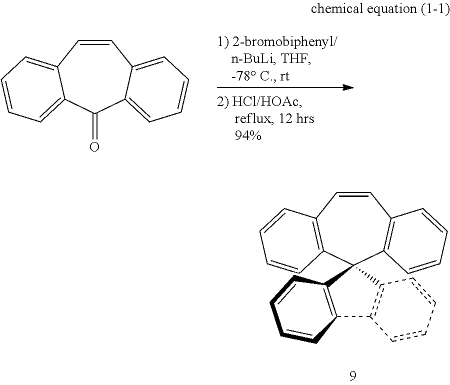

### Example 2: Synthesis of Compound 10 (Spiro-fluorene-dibenzosuberan-10,11-dione)

A stir bar was placed into a 250 ml single-neck round-bottom flask, and the flask was then equipped to a reflux condenser system. The compound 9 (10.27 g, 30 mmol), benzene-seleninic anhydride (BSA, 12.96 g, 36 mmol) and chlorobenzene (180 ml) were sequencially added to the round-bottom flask. The mixture was refluxed for 18 hours. The residual chlorobenzene was then removed through distillation under reduced pressure. The crude product was then purified by column chromatography using a mixture of dichloromethane and n-hexane (1:1) as eluent and compound 10 (9.65 g, yield: 86%) was obtained. The above reaction is represented by the chemical equation (1-2).

Spectral data as follow: Tₘ 245° C. (DSC); M.W.: 372.41; ¹H NMR (400 MHz, CDCl₃) δ 7.83 (dd, J=7.7, 1.8, 4H), 7.39 (td, J=7.5, 0.8, 2H), 7.34 (td, J=7.3, 0.8, 2H), 7.24-7.16 (m, 4H), 7.03 (d, J=7.7, 2H), 6.59 (d, J=8.3, 2H); ¹³C NMR (100 MHz, CDCl₃) δ 199.1, 154.4, 142.8, 139.6, 134.1, 133.5, 130.7, 129.4, 129.0, 128.1, 128.0, 125.5, 120.7, 67.9; MS (FAB) 373.1 (M⁺, 100): TLC R_{f} 0.3 (dichloromethane/hexane, 1/1); HR-MS Anal. calcd for C₂₇H₁₆O₂: 372.1150, found: 372.1153; Anal. Calcd for C₂₇H₁₆O₂: C, 87.08, H, 4.33. Found: C, 87.14, H, 4.27.

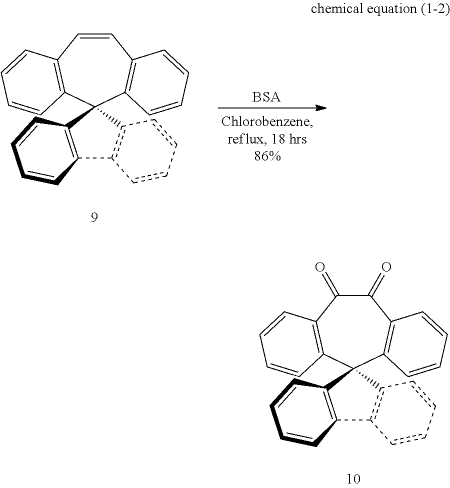

### Example 3: Synthesis of Compound 11

A stir bar was placed into a 500 ml round-bottom flask, which was then equipped to a reflux condenser system. The compound 10 (8.94 g, 24 mmol), benzene-1,2-diamine (3.11 g, 28.8 mmol) and the catalyst 4-methylbenzenesulfonic acid (228 mg, 1.2 mmol) were sequentially added to the flask. After adding with 120 ml of chloroform, the mixture was refluxed for 12 hours. The flask was then removed from the reflux system and cooled down to room temperature. The reaction mixture was then extracted with dichloromethane (3×150 ml). The combined extractant was then dried by adding anhydrous magnesium sulfate, followed by filtration and condensation with a rotary evaporator to obtain the compound 11 (10.19 g, yield: 95%). The above reaction is represented by the chemical equation (1-3).

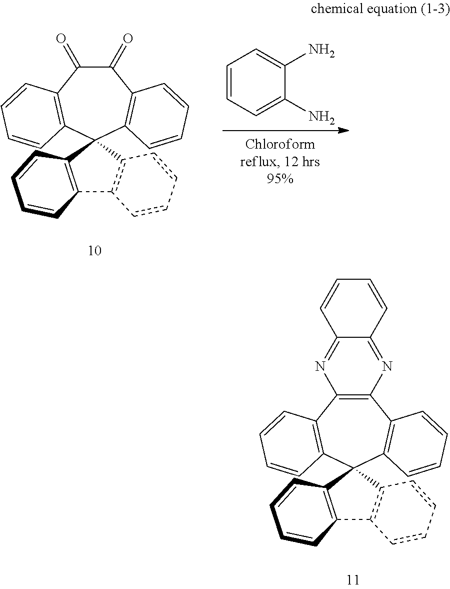

Spectral data as follow: Tₘ 267° C. (DSC); M.W.: 444.52; ¹H NMR (400 MHz, CDCl₃) δ 8.39 (dt, J=7.6, 0.8, 2H), 8.30 (q, J=3.0, 2H), 7.88 (q, J=3.0, 2H), 7.74 (d, J=7.5, 2H), 7.47-7.43 (m, 2H), 7.31 (bs, 2H), 7.20 (d, J=3.3, 4H), 7.02 (bs, 2H), 6.68 (bs, 2H); ¹³C NMR (100 MHz, CDCl₃) δ 153.2, 149.3, 145.7, 141.8, 140.1, 138.0, 133.3, 130.2, 129.5, 129.4, 128.4, 127.9, 127.8, 127.1, 120.4, 66.4; MS (MALDI-TOF) 445.2 (M+H⁺, 100); TLC R_{f} 0.4 (dichloromethane/hexane, 1/1); HR-MS Anal. calcd for C₃₃H₂₁N₂: 445.1699, found: 445.1656.

### Example 4: Synthesis of Compound 12

A stir bar was placed into a 25 ml single-neck round-bottom flask, which was then equipped to a reflux condenser system, followed by drying under vacuum. The compound 11 (444 mg, 1 mmol), IrCl₃H₂O (99 mg, 0.34 mmol) and 2-ethoxyethanol (7 ml) were sequentially added to the flask. The reaction mixture was refluxed for 24 hours, and the flask was then removed from the reflux system and cooled down to room temperature. 10 ml of deionized water was then added to the flask so that brown solid precipitates were obtained. The brown precipitates were collected with a suction funnel and then washed by deionized water. After being washed by a few ethanol for three times, the precipitates were then dried in an oven to obtain the intermediate product as a brown solid. A stir bar was placed into another 25 ml single-neck round-bottom flask, and was then equipped on a reflux condenser system. The intermediate product, 2,4-pentadione (51 mg, 0.51 mmol), sodium carbonate (180 mg, 1.7 mmol) and 2-ethoxyethanol (8.5 ml) were sequentially added to the flask. The reaction mixture was refluxed for 12 hours, and the flask was then removed from the reflux system and cooled down to room temperature. 10 ml of deionized water was then added to the flask to obtain grey solid precipitates. The grey solid precipitates were collected with a suction funnel and then washed by deionized water. After being washed by a small amount of ethanol for three times, the crude product was purified by column chromatography by using a mixture of dichloromethane and n-hexane (1:1) as eluent to obtain the compound 12 (i.e., the compound of chemical formula (1): Ir(QSTIF)₂(acac), yield: 17%). The above reaction is represented by the chemical equation (1-4).

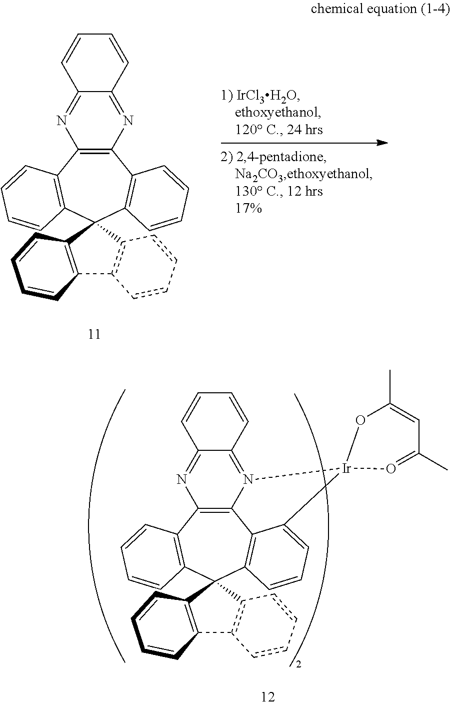

Spectral data as follow: M.W.: 1178.82; ¹H NMR (400 MHz, CDCl₃) δ 8.25 (d, J=8.2, 2H), 8.20 (d, J=8.8, 2H), 8.03 (d, J=6.6, 2H), 7.83 (d, J=7.8, 2H), 7.76-7.70 (m, 4H), 7.57 (d, J=7.4, 2H), 7.51 (q, J=7.2, 6H), 7.43 (t, J=7.0, 2H), 7.22 (t, J=7.6, 2H), 7.12-7.07 (m, 4H), 6.74 (t, J=7.3, 2H), 6.68 (d, J=7.8, 2H), 6.57 (d, J=8.0, 2H), 6.38 (d, J=7.4, 2H), 6.23 (t, J=7.7, 2H), 4.59 (s, 1H), 1.48 (s, 6H); ¹³C NMR (100 MHz, CDCl₃) δ 186.0, 164.2, 156.1, 153.5, 151.4, 150.5, 146.3, 144.1, 143.4, 141.5, 141.2, 140.3, 138.5, 137.3, 137.0, 133.6, 130.4, 130.3, 129.7, 129.2, 129.2, 128.1, 128.1, 127.8, 127.7, 127.3, 126.8, 125.8, 124.2, 123.2, 120.6, 120.2, 100.0, 66.5, 28.0; MS (MALDI-TOF) 1179.4 (M+H⁺, 3); TLC R_{f} 0.25 (dichloromethane/hexane, 1/1); HR-MS Anal. calcd for C₇₁H₄₆IrN₄O₂: 1179.3250, found: 1179.3810.

### Example 5: Synthesis of Compound 13

A stir bar was placed into a 500 ml tri-neck flask. A reflux system and a addition funnel were installed and the flask was then dried by vacuum. 5H-Dibenzosuberenone (21.72 g, 120 mmol) were diluted in 250 ml of tetrahydrofuran and then added to flask for reflux. During reflux, 3M methylmagnesium chloride (48 ml, 144 mmol) tetrahydrofuran solution was first added to the addition funnel and added into the tri-neck flask dropwise. The reaction mixture was reflux for 18 hours, and then the flask with the reaction mixture was cooled down to room temperature. 80 ml of saturated ammonium chloride was then dropwise added to the reaction mixture on an ice bath. The precipitates were removed by filtration, followed by removing the solvent by rotatory evaporation. The resultant product was then added with a mixture of ethyl acetate (20 ml) and hexane (200 ml), followed by stirring. The resultant white solids were then collected with a suction funnel to obtain the compound 13 as a white solid (20.38 g, yield: 76%). The above reaction is represented by the chemical equation (2-1).

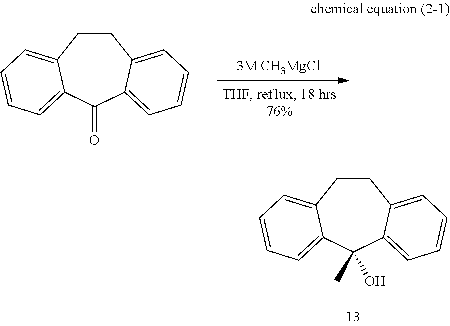

Spectral data as follow: M.W.: 224.30; 1H NMR (400 MHz, CDCl₃) δ 7.94 (d, J=7.9, 2H), 7.25 (t, J=7.1, 2H), 7.19 (t, J=7.3, 2H), 7.11 (t, J=7.3, 2H), 3.35-3.27 (m, 2H), 3.12-3.05 (m, 2H), 2.19 (s, 2H), 1.91 (s, 3H); ¹³C NMR (100 MHz, CDCl₃) δ 145.7, 139.2, 130.2, 127.3 126.4, 34.7, 34.7; TLC R_{f}0.27 (dichloromethane/hexane, 1/1); HR-MS (EI) Anal. Calcd for C₁₆H₁₆O: 224.1196, found: 224.1192.

### Example 6: Synthesis of Compound 14

A stir bar was placed into a 100 ml single-neck flask. The single-neck flask was then installed to a reflux condenser system, followed by being dried by vacuum. The compound 13 (20.0 g, 89 mmol) and acetic anhydride (45.5 ml, 446 mmol) were added to the flask and the reaction mixture was then refluxed for 24 hours. The residual acetic anhydride was removed from the reaction mixture through distillation under reduced pressure. The reaction mixture was then extracted by dichloromethane (3×150 ml) and added with anhydrous magnesium chloride (about 3.0 g), followed by filtration and condensation with a rotary evaporator to obtain a crude product. The crude product was then purified by column chromatography using hexane as eluent to obtain the compound 14 (13.57 g, yield: 74%). The above reaction is represented by the chemical equation (2-2).

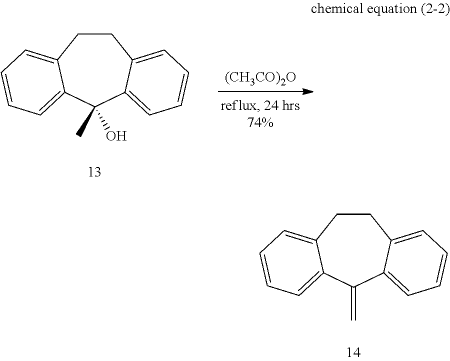

Spectral data as follow: M.W.: 206.28; ¹H NMR (400 MHz, CDCl₃) δ 7.35 (dd, J=7.2, 1.2, 2H), 7.20 (td, J=7.4, 1.5, 2H), 7.17 (td, J=6.4, 1.6, 2H), 7.12 (dd, J=7.0, 1.7, 2H), 5.41 (s, 2H), 3.15 (s, 4H); ¹³C NMR (100 MHz, CDCl₃) δ 151.8, 141.1, 138.3, 128.9, 128.1, 127.6, 126.2, 117.4, 33.3; TLC R_{f} 0.6 (hexane); HR-MS (EI) Anal. Calcd for C₁₆H₁₄: 206.1090, found: 206.1089.

### Example 7: Synthesis of Compound 15

A stir bar was placed into a 100 ml dual-neck round-bottom flask. The dual-neck flask was then installed to a reflux condenser system, followed by being dried by vacuum. The compound 13 (2.5 g, 12 mmol), anhydrous tetrafurane (20 ml) and anisole (20 ml) were added to the flask, followed by adding lithium aluminum hydride (1.0 g, 26 mmol). The reaction mixture was refluxed for 2.5 hours, and then the flask with the reaction mixture was cooled down to room temperature to obtain the crude product. To the crude product was then drop-wise added with aqueous hydrogen chloride solution (1N, 100 ml) on an ice bath, followed by being extracted with dichloromethane (3×100 ml). The combined extractant was then added with anhydrous magnesium sulfide (about 1.0 g) to dry, followed by filtration and condensation with a rotary evaporator to obtain another crude product. The resulted crude product was then purified by column chromatography using hexane as eluent to obtain the compound 15 (2.1 g, yield: 79%). The above reaction is represented by the chemical equation (2-3).

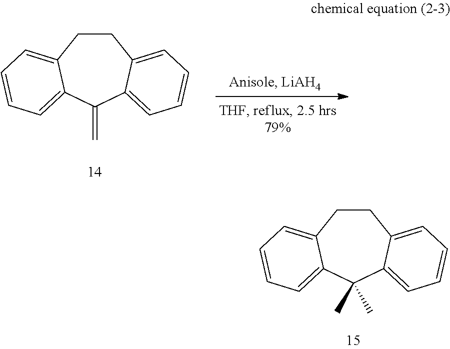

Spectral data as follow: M.W.: 222.32; ¹H NMR (400 MHz, CDCl₃) δ 7.50-7.48 (m, 2H), 7.12-7.09 (m, 6H), 3.35 (s, 4H), 1.92 (s, 6H); ¹³C NMR (100 MHz, CDCl₃) δ 146.6, 139.7, 131.1, 126.4, 125.9, 125.7, 33.9, 31.9; TLC R_{f}0.5 (hexane); HR-MS (EI) Anal. Calcd for C₁₇H₁₈: 222.1403, found: 222.1392.

### Example 8: Synthesis of Compound 16

A stir bar was placed into a 25 ml single-neck round-bottom flask, which was then installed to a reflux condenser system, followed by being dried by vacuum. The compound 15 (714 mg, 3.2 mmol), N-bromosuccinimide (598 mg, 3.36 mmol), and carbon tetrachloride (12 ml) were sequentially added to the flask, followed by adding benzoyl peroxide (22 mg, 0.07 mmol). The reaction mixture was refluxed for 2.5 hours, and then cooled down to room temperature. Saturated aqueous sodium bisulfite solution (30 ml) was then added to quench the reaction. The reaction mixture was then extracted with dichloromethane (3×50 ml) and then added with anhydrous magnesium sulfate (about 5.0 g) to dry, followed by filtration and condensation with a rotary evaporator to obtain a crude product. The crude product was then purified by neutral alumina column chromatography using n-hexane as eluent to obtain the compound 16 as transparent liquid (686 mg, yield: 97%). The above reaction is represented by the chemical equation (2-4).

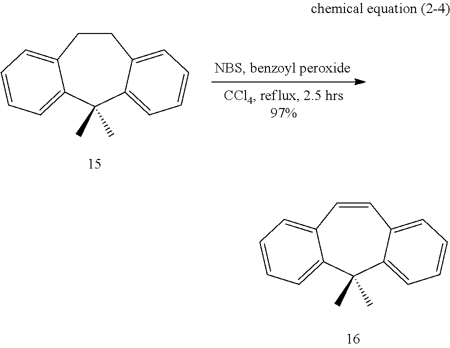

Spectral data as follow: M.W.: 220.31; ¹H NMR (400 MHz, CDCl₃) δ 7.54 (dd, J=7.5, 1.0, 2H), 7.36-7.33 (m, 4H), 7.22 (td, J=8.0, 1.0, 2H), 7.06 (s, 2H); ¹³C NMR (100 MHz, CDCl₃) δ 144.3, 134.9, 132.2, 129.9, 128.5, 125.6, 124.8, 41.0; TLC R_{f}0.5 (hexane); HR-MS (EI) Anal. Calcd for C₁₇H₁₆: 220.1247, found: 220.1239.

### Example 9: Synthesis of Compound 17

A stir bar was placed into a 50 ml single-neck round-bottom flask, which was then installed to a reflux condenser system. The compound 16 (679 mg, 3.1 mmol), benzene-seleninic anhydride (BSA, 1663 mg, 4.6 mmol) and chlorobenzene (18 ml) were sequentially added to the round-bottom flask. The reaction mixture was refluxed for 96 hours. The chlorobenzene was then removed through distillation under reduced pressure. The crude product was then purified by column chromatography using a mixture of dichloromethane and n-hexane (1:1) as eluent and compound 17 (356 mg, yield: 46%) was obtained. The above reaction is represented by the chemical equation (2-5).

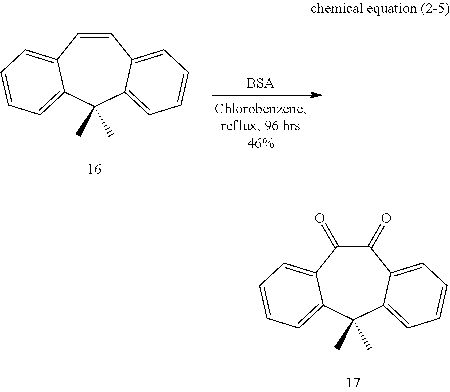

Spectral data as follow: M.W.: 250.29; ¹H NMR (400 MHz, CDCl₃) 7.86 (d, J=7.6, 2H), 7.53-7.47 (m, 4H), 7.35 (td, J=6.8, 1.5, 2H), 1.91 (s, 6H); ¹³C NMR (100 MHz, CDCl₃) δ 187.4, 148.7, 135.0, 133.1, 133.1, 127.4, 124.4, 42.3, 34.3; TLC R_{f} 0.16 (dichloromethane/hexane, 1/1); HR-MS (EI) Anal. Calcd for C₁₇H₁₄NaO₂ (M+Na⁺): 273.0886, found: 273.0888.

### Example 10: Synthesis of Compound 18

A stir bar was placed into a 50 ml round-bottom flask, which was then installed to a reflux condenser system. The compound 17 (346 mg, 1.38 mmol), benzene-1,2-diamine (179 mg, 1.66 mmol) and the catalyst 4-methylbenzenesulfonic acid (13 mg, 0.07 mmol) were sequentially added to the flask. After adding with 14 ml of chloroform, the reaction mixture was refluxed for 12 hours. The flask was then removed from the reflux system and cooled down to room temperature. The reaction mixture was then extracted with dichloromethane (3×50 ml). The combined extractant was then dried by adding anhydrous magnesium sulfate, followed by filtration and condensation with a rotary evaporator to remove the solvent and to obtain a crude product. The crude product was then purified by by column chromatography using a mixture of dichloromethane and n-hexane (1:1) as eluent and compound 18 (412 mg, yield: 93%) was obtained. The above reaction is represented by the chemical equation (2-6).

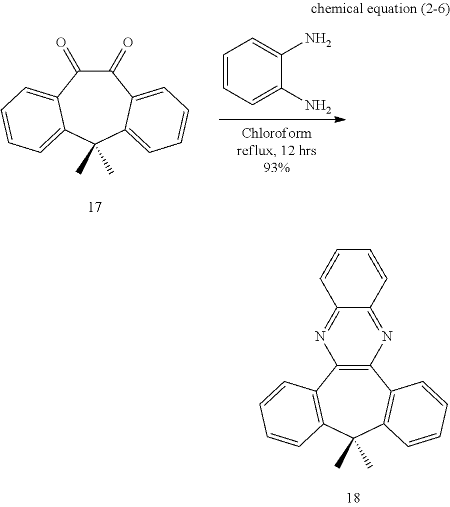

Spectral data as follow: M.W.: 322.40; ¹H NMR (400 MHz, CDCl₃) δ 8.26 (qd, J=3.1, 1.2, 2H), 8.13 (dt, J=7.3, 1.3, 2H), 7.82 (qd, J=2.9, 0.8, 2H), 7.58 (d, J=7.8, 2H), 7.47-7.38 (m, 4H), 2.06 (s, 3H), 1.20 (s, 3H); ¹³C NMR (100 MHz, CDCl₃) δ 153.3, 149.6, 141.4, 135.8, 132.5, 129.9, 129.7, 129.3, 126.8, 123.7, 41.6, 30.1, 28.2; TLC R_{f} 0.27 (dichloromethane/hexane, 1/2); HR-MS Anal. calcd for C₂₃H₁₇N₂: 321.1386, found: 321.1377.

### Example 11: Synthesis of Compound 19

A stir bar was placed into a 100 ml single-neck round-bottom flask, which was then installed to a reflux condenser system, followed by being dried by vacuum. The compound 18 (2.4 g, 7.5 mmol), IrCl₃H₂O (748 mg, 2.5 mmol) and 2-ethoxyethanol (50 ml) were sequentially added to the flask. The flask was then put in an oil bath at 120° C. for reflux. The reaction mixture was refluxed for 24 hours, and then the flask was removed from the reflux system and cooled down to room temperature. The solvent in the reaction mixture was then removed by a rotary evaporator. 10 ml of deionized water was then added to the flask to obtain solid precipitates. The solid precipitates were collected with a suction funnel and then washed by deionized water. After being washed by a small amount of ethanol, the precipitates were then dried in an oven to obtain the intermediate product as a brown solid. A stir bar was placed into another 100 ml round-bottom flask, which was then installed to a reflux condenser system, followed by being dried by vacuum. The intermediate product obtained above, 2,4-pentadione (375 mg, 3.75 mmol), sodium carbonate (1.32 g, 12.5 mmol) and 2-ethoxyethanol (63 ml) were sequentially added to the flask. The flask was then put in an oil bath at 130° C. for reflux. The reaction mixture was refluxed for 18 hours, and then the flask was removed from the reflux system and cooled down to room temperature. The solvent was then removed by a rotary evaporator. 100 ml of deionized water was then added to the flask to obtain solid precipitates. The solid precipitates were collected with a suction funnel and then washed by deionized water. After being washed by ethanol for three times, the crude product was purified by column chromatography using a mixture of dichloromethane and n-hexane (1:1) as eluent to obtain the compound 19 (i.e., the compound of chemical formula (2): Ir(DMQSTI)₂(acac), (454 mg, yield: 19%). The above reaction is represented by the chemical equation (2-7).

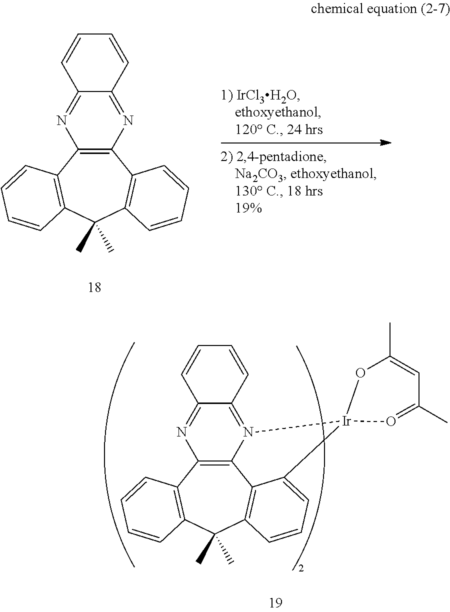

Spectral data as follow: M.W.: 934.11; ¹H NMR (400 MHz, CDCl₃) δ 8.20 (t, J=8.4, 4H), 7.90 (dt, J=8.0, 1.3, 2H), 7.69-7.64 (m, 4H), 7.52 (td, J=7.8, 1.5, 2H), 7.19 (t, J=4.4, 2H), 6.74 (d, J=3.9, 4H), 4.43 (s, 4H), 2.03 (s, 6H), 1.41 (s, 6H), 1.28 (s, 6H); ¹³C NMR (100 MHz, CDCl₃) δ 185.5, 164.2, 153.5, 152.8, 150.3, 148.1, 142.2, 141.3, 140.1, 135.3, 134.8, 132.5, 129.8, 129.6, 129.1, 128.8, 126.8, 125.9, 124.5, 128.2, 99.6, 53.4, 41.5, 32.1, 28.3, 27.8; TLC R_{f} 0.2 (dichloromethane/hexane, 3/2); HR-MS Anal. calcd for C₅₁H₄₁N₄O₂Ir: 934.2853, found: 934.2830.

Evaluation of the Series of the Iridium (III) Complex Compounds with Cyclic Quinoxaline-Fused Dibenzosuberene Ligands as Guest Materials

The compounds of chemical formulas (1) and (2), synthesized through the protocols provided as Example 1 to Example 4 and Example 5 to Example 11, respectively, were evaluated for their thermal, photophysical, and electrochemical properties, such as their wavelengths of maximum absorption (Abs. λmax), wavelengths of maximum emission (Em, λmax), full width at half maximum (FWHM), quantum yield (op), oxidation potential (Eox), reduction potential (Ered), the highest occupied molecular orbital (EHOMO), the lowest occupied molecular orbital (ELUMO), energy gap (Eg), the triplet energy level (ET) and the decomposition temperature (Td). Those properties mentioned above were measured according to the following methods and conditions.

The wavelengths of maximum absorpotion (Abs. λₘₐₓ), the wavelengths of maximum emission (Em, λₘₐₓ), and full width at half maximum (FWHM) were measured in a solution using dichloromethane as the solvent. Quantum yield (Φₚ) was measured with Hamamatsu C9920. The triplet energy level measured at low temperature by spectrometer is the basis of selecting the guest material of phosphorescent emitter. The decomposition temperature was measured by thermogravimetric analyzer (TGA) and are considered to be the basis of the thermal stability for the device fabrication and optoelectronic performance.

The electrochemical properties, including Eₒₓ, E_{red}, E_{HOMO}, E_{LUMO}, E_{g}, and E_{T}, were measured by way of cyclic voltammetry (CV) in a solution using dichloromethane as a solvent. Platinum wire electrode was used as a counter electrode and glassy carbon electrode was used as a working electrode and Ag/AgCl as a reference electrode. Ferrocene was used as a standard. The CV curves were calibrated using the ferrocene/ferrocenium (Fc/Fc⁺) redox couple as an external standard which was measured under same condition before and after the

TABLE 1

| | Chemical | | |
|---|---|---|---|
| compound | formula (1) | Chemical formula (2) | Compound 11 |
| Abs. λₘₐₓ (nm) | 388, 485 | 383, 485 | 273, 352 |
| Em, λₘₐₓ (nm) | 686 | 676 | 407 |
| FWHM (nm) | 64 | 50 | — |
| Φₚ (%) | 12.7 | 6.5 | — |
| Eₒₓ (V) | 0.55 | 0.6 | 1.18 |
| E_{red} (V) | −1.75, −2.11 | −1.82, −2.13 | −2.04, −2.59 |
| E_{HOMO} (eV) | −5.4 | −5.35 | −5.98 |
| E_{LUMO} (eV) | −3.1 | −3.0 | −2.76 |
| E_{g} (eV) | 2.3 | 2.35 | 3.22 |
| E_{T} (eV) | 1.81 | 1.84 | — |
| T_{d} (° C.) | 361 | 371 | 357 |

measurement of samples. The energy level of Fc/Fc+ was assumed at −4.8 eV to vacuum. The energy level of LUMO (E_{LUMO}) was obtained by adding the bandgap energy (E_{g}) to the HOMO energy level (E_{HOMO}).

Those properties as mentioned above of the compounds with the structures of chemical formula (1): Ir(QSTIF)₂(acac) and chemical formula (2): Ir(DMQSTI)₂(acac) are shown in Table 1.

According to Table 1, the absorption spectra of the chemical formula (1): Ir(QSTIF)₂(acac) are very similar with those of the chemical formula (2): Ir(DMQSTI)₂(acac). The absorption peaks appear at around 380 nm for both compound which correspond to their ligand-centered (C—N chelating group) π-π* transitions. The absorption peaks at 485 nm are resulted from the spin-allowed singlet metal-to-ligand charge transfer (S₀→¹MLCT) and those at around 600 nm are resulted from the spin-forbidden triplet metal-to-ligand charge transfer (S₀→³MLCT). In addition, the decomposition temperatures of the compounds of chemical formula (1) and chemical formula (2) are both higher than 350 □ (chemical formula (1) is 361° C. and chemical formula (2) is 371° C.), so that the decomposition caused by the heat is not easily occurred during the thermal vacuum deposition process. Based on the reasons mentioned above, such series of iridium complex compounds have excellent thermal stability and suitable triplet energy levels, and are quite beneficial to be the red-emitting guest materials of an organic luminescent unit.

The oxidation potentials of the compounds of chemical formula (1) (Ir(QSTIF)₂(acac)) and chemical formula (2) (Ir(DMQSTI)₂(acac)) are +0.61 V and +0.55 V, respectively. The oxidation of such category of iridium complex compounds are resulted from the oxidation of their Ir(III) to Ir(IV). The cyclic ligand DimethylQSTI (i.e., the moiety DMQSTI with the structure of compound 18) of the compound of chemical formula (2) has a higher reduction potential than the ocyclic ligand QSTIF (i.e., the moiety with the structure of compound 11) of the compound of chemical formula (1) does. In other words, the ligand DMQSTI of the compound of chemical formula (2) is a stronger electron donor. Therefore, comparing with the compound of the compound of chemical formula (1), the iridium in the compound of chemical formula (2) (Ir(DMQSTI)₂(acac)) is easier to be oxidized after the ligand DMQSTI chelating with the iridium center, and thus the compound of chemical formula (2) (Ir(DMQSTI)₂(acac)) has a lower oxidation potential.

On the other hand, each of the compounds of chemical formula (1) and chemical formula (2) have two sets of reduction potentials, −1.75 V/−2.11 V and −1.82 V/−2.13 V, respectively. The first reduction potential of the compound of chemical formula (1) (−1.75 V) and chemical formula (2) (−1.82 V) are attributable to the heterocyclic moiety of the C—N chelating group as an electron acceptor. As mentioned above, comparing to the chemical formula (1), the chemical formula (2) has a stronger electron-donor ligand DMQSTI, and therefore the chelating group of the chemical formula (2) is harder to be reduced.

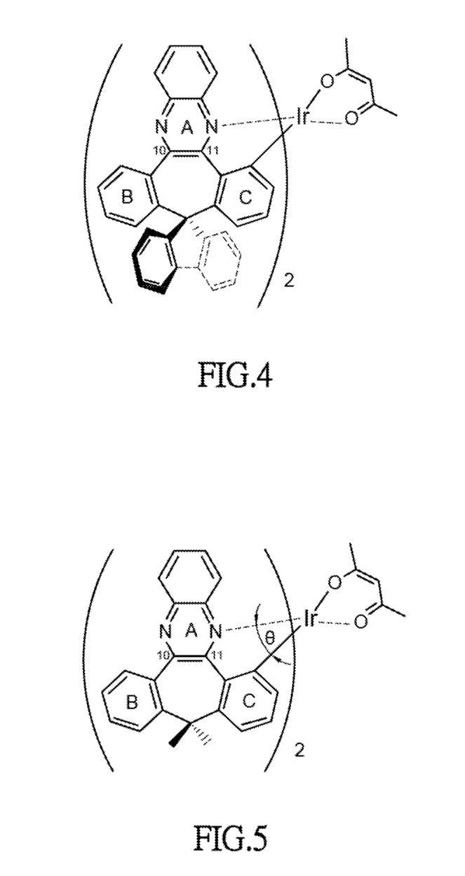

Please refer to Table 2 and FIGS. 4 and 5. Dihedral angles of the compounds of chemical formula (1) and chemical formula (2) are shown in Table 2. FIG. 4 depicts the chemical structure of the compound of Chemical Formula (1): Ir(QSTIF)₂(acac) according to one embodiment of the invention. FIG. 5 depicts the chemical structure of the compound of Chemical Formula (2): Ir(DMQSTI)₂(acac) according to one embodiment of the invention. The 3D structures of the compounds of chemical formula (1) and chemical formula (2) are identified by X-ray crystallographic analysis. As shown in Table 2 and FIG. 4, in chemical formula (1), the dihedral angles between the benzene ring B/benzene ring C and the double bond C₁₀═C₁₁ in the central seven-membered ring are 29.3° and 27.6°, respectively, and the dihedral angle between the quinoxaline ring A and the double bond C₁₀═C₁₁ in the central seven-membered ring is 25.5°. The nonplanar configuration of the DMQSTI ligand of chemical formula (2) may be attributable to the fusion of quinoxaline to the central seven-membered ring, which resulted in the twist of the central seven-membered ring.

Similarly, as shown in Table 2 and FIG. 5, in chemical formula (2), the dihedral angles between the quinoxaline ring A and the benzene ring B/benzene ring C flanking the central seven-membered core moiety are 46.6° and 25.2°, respectively. The difference between the two aforementioned dihedral angles may be attributable to the twisted central seven-membered ring moiety, which is resulted from the steric hinderance caused by the

TABLE 2

| | Chemical Formula (1): | Chemical Formula (2): |
|---|---|---|
| Compound | Ir(QSTIF)₂(acac) | Ir(DMQSTI)₂(acac) |
| Ring A and C₁₀ = C₁₁ | 25.5° | — |
| Ring B and C₁₀ = C₁₁ | 29.3° | — |
| Ring C and C₁₀ = C₁₁ | 27.6° | — |
| Ring A and Ring B | — | 46.6° |
| Ring A and Ring C | — | 25.2° |

di-methyl moiety. However, the lesser dihedral angle between the quinoxaline ring A and the benzene ring C which forms the chelating bond with the iridium ion is attributable to the ligand configuration. The di-methyl moiety actually contributes to the nonplanar 3D structure of the ligand DMQSTI, and to prevent concentration-quenching effect resulted from intermolecular stacking. In chemical formula (2), the angle θ of the chelating bond C—Ir—N is 80°. However, the angle θ of the chelating bond C—Ir—N is 86° in the Ir(ppy)₃ molecule which is a classical green phosphorescent material.

The Device Efficiency for Compounds (Chemical Formula (1): Ir(QSTIF)₂(Acac) and Chemical Formula (2): Ir(DMQSTI)₂(Acac)) which were Used in Organic Light Emitting Diodes

A. Fabrication of Red Phosphorescent OLEDs Using the Compounds of Chemical Formula (1): Ir(QSTIF)₂(Acac) and Chemical Formula (2): Ir(DMQSTI)₂(Acac) as Guest Materials Through Solution Processes

The structures from unit 1 to unit 12 are ITO/PEDOT:PSS (35 nm)/host:guest (15 nm)/TPBI (40 nm)/LiF (1 nm)/Al (100 nm). The structures from unit 16 to unit 27 are ITO/PEDOT:PSS (35 nm)/host:guest (25 nm)/TPBI (40 nm)/LiF (1 nm)/Al (150 nm). The host materials of the organic luminescent layer are 4,4′-Bis(N-carbazolyl)-1,1′-biphenyl (CBP), N,N′-Di(1-naphthyl)-N,N′-diphenyl-(1,1′-biphenyl)-4,4′-diamine (NBP), 3,5-di(9H-carbazol-9-yl)tetraphenylsilane (SimCP2), 4,4′,4″-tri(N-carbazolyl)triphenylamine (TCTA), or 2,7-bis(carbazo-9-yl)-9,9-ditolyfluorene (Spiro-2CBP). In addition, a co-host system is also employed through doping 10 wt % of Bis(10-hydroxybenzo[h]quinolinato)beryllium (Bebq₂) in NBP. The guest materials of the organic luminescent layer are based on the compounds of Chemical Formula (1) and Chemical Formula (2). The host materials were mixed with the guest materials at various ratios. Herein, the material of first electrode layer of the organic electroluminescent device is ITO. The material of the second electrode layer is aluminum with the thickness of 100 nm. The material of the hole injection layer is PEDOT:PSS with the thickness of 35 nm. The thickness of the organic luminescent layer is 15 nm. The material of the electron transporting layer is TBPI with the thickness of 40 nm. The material of electron injecting layer is LiF with the thickness of 1 nm. The fabrication process of the OLED units in the present experimental example includes first spin-coating an aqueous solution of PEDOT:PSS to form a HIL on a pre-cleaned ITO anode. Before depositing the following organic luminescent layer, the solution was prepared by dissolving the host and guest materials at various ratios in THF solvent. The resulting solution was then spin-coated. Followed by the TPBi, LiF, and Al, were thermally deposited under vacuum.

B. Fabrication of Red Phosphorescent OLEDs Using the Compound of Chemical Formula (2): Ir(DMQSTI)2(Acac) as Guest Materials Through Dry Processes

The unit structure is ITO/HATCN (2.5 nm)/TAPC (40 nm)/host:guest (20 nm)/TPBI (30 nm)/LiF (1 nm)/Al (100 nm). The host material of the organic luminescent layer is WPH401, as represented by Chemical Formula (3).

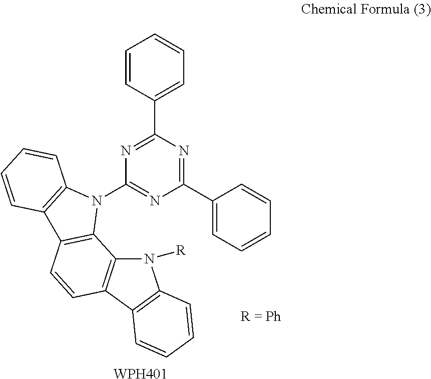

The guest materials of the organic luminescent layer are based on the compounds of Chemical Formula (2). The host materials were mixed with the guest materials at various ratios. Herein, the material of first electrode layer of the organic electroluminescent device is ITO. The material of the second electrode layer is aluminum with the thickness of 100 nm. The material of the hole injection layer is 2,3,6,7,10,11-Hexacyano-1,4,5,8,9,12-hexaazatriphenylene (HATCN) with the thickness of 2.5 nm. The thickness of the organic luminescent layer is 20 nm. The material of the hole transporting layer is Bis[4-[N,N′-di(p-tolyl)amino]phenyl]cyclohexane (TAPC) with the thickness of 30 nm. The material of the electron transporting layer is TBPI with the thickness of 30 nm. The material of electron injecting layer is LiF with the thickness of 1 nm. The units in the present experimental example were prepared by coevaporation method to form each layer thereof.

C. Efficiency Evaluation of the Organic Luminescent Units Fabricated as Above

The organic luminescent units fabricated through aforementioned processes were evaluated for their wavelengths of maximum emission (Em, λₘₐₓ), turn-on voltages (Vₒₙ) at 1 cd/m², current efficiencies ile (cd/A) at 100 cd/m² and maximum current efficiencies, power efficiencies ηₚ, (lm/W) at 100 cd/m² and maximum power efficiencies, external quantum efficiencies ηₙₑₓₜ (EQE, %) at 100 cd/m² and maximum external quantum efficiencies, and CIExy. The device configurations of each OLED units are shown in Table 3 and the characteristics of these OLED units are shown in Table 4.

TABLE 3

| | Second | Electron | Electron | | | | First |
|---|---|---|---|---|---|---|---|
| Unit | electrode | injection | transport | Host | Guest (wt %) | | electron |
| | | | | | | HIL | |
| 1 | Al | LiF | TBPI | CBP | Ir(QSTIF)₂(acac) 7% | PEDOT:PSS | ITO |
| 2 | Al | LiF | TBPI | CBP | Ir(QSTIF)₂(acac) 10% | PEDOT:PSS | ITO |
| 3 | Al | LiF | TBPI | CBP | Ir(QSTIF)₂(acac) 15% | PEDOT:PSS | ITO |
| 4 | Al | LiF | TBPI | NPB/Bebq₂ | Ir(QSTIF)₂(acac) 7% | PEDOT:PSS | ITO |
| 5 | Al | LiF | TBPI | NPB/Bebq₂ | Ir(QSTIF)₂(acac) 10% | PEDOT:PSS | ITO |
| 6 | Al | LiF | TBPI | NPB/Bebq₂ | Ir(QSTIF)₂(acac) 15% | PEDOT:PSS | ITO |
| 7 | Al | LiF | TBPI | CBP | Ir(DMQSTI)₂(acac) 7% | PEDOT:PSS | ITO |
| 8 | Al | LiF | TBPI | CBP | Ir(DMQSTI)₂(acac) 10% | PEDOT:PSS | ITO |
| 9 | Al | LiF | TBPI | CBP | Ir(DMQSTI)₂(acac) 15% | PEDOT:PSS | ITO |
| 10 | Al | LiF | TBPI | NPB/Bebq₂ | Ir(DMQSTI)₂(acac) 7% | PEDOT:PSS | ITO |
| 11 | Al | LiF | TBPI | NPB/Bebq₂ | Ir(DMQSTI)₂(acac) 10% | PEDOT:PSS | ITO |
| 12 | Al | LiF | TBPI | NPB/Bebq₂ | Ir(DMQSTI)₂(acac) 15% | PEDOT:PSS | ITO |
| | | | | | | HT/HIL | |
| 13 | Al | LiF | TBPI | WPH401 | Ir(DMQSTI)₂(acac) 5% | TAPC/HATCN | ITO |
| 14 | Al | LiF | TBPI | WPH401 | Ir(DMQSTI)₂(acac) 10% | TAPC/HATCN | ITO |
| 15 | Al | LiF | TBPI | WPH401 | Ir(DMQSTI)₂(acac) 15% | TAPC/HATCN | ITO |
| | | | | | | HIL | |
| 16 | Al | LiF | TBPI | SimCP2 | Ir(QSTIF)₂(acac) 15% | PEDOT:PSS | ITO |
| 17 | Al | LiF | TBPI | TCTA | Ir(QSTIF)₂(acac) 15% | PEDOT:PSS | ITO |
| 18 | Al | LiF | TBPI | Spiro-2CBP | Ir(QSTIF)₂(acac) 5% | PEDOT:PSS | ITO |
| 19 | Al | LiF | TBPI | Spiro-2CBP | Ir(QSTIF)₂(acac) 7% | PEDOT:PSS | ITO |
| 20 | Al | LiF | TBPI | Spiro-2CBP | Ir(QSTIF)₂(acac) 10% | PEDOT:PSS | ITO |
| 21 | Al | LiF | TBPI | Spiro-2CBP | Ir(QSTIF)₂(acac) 15% | PEDOT:PSS | ITO |
| 22 | Al | LiF | TBPI | Spiro-2CBP | Ir(QSTIF)₂(acac) 20% | PEDOT:PSS | ITO |
| 23 | Al | LiF | TBPI | NPB | Ir(QSTIF)₂(acac) 7% | PEDOT:PSS | ITO |
| 24 | Al | LiF | TBPI | NPB | Ir(QSTIF)₂(acac) 10% | PEDOT:PSS | ITO |
| 25 | Al | LiF | TBPI | NPB | Ir(QSTIF)₂(acac) 15% | PEDOT:PSS | ITO |
| 26 | Al | LiF | TBPI | NPB | Ir(QSTIF)₂(acac) 20% | PEDOT:PSS | ITO |
| 27 | Al | LiF | TBPI | — | Ir(QSTIF)₂(acac) 100% | PEDOT:PSS | ITO |

TABLE 4

| | | | | ηₑₓₜ (%) | | η_{c} (cd/A) | | ηₚ (lm/W) | |
|---|---|---|---|---|---|---|---|---|---|
| Unit | Em, λₘₐₓ | CIE (x, y) | Vₒₙ (V) | 100 cd/m² | max | 100 cd/m² | max | 100 cd/m² | max |
| 1 | 688 | (0.66, 0.25) | 5 | 6.8 | 7.2 | 0.31 | 0.39 | 0.15 | 0.2 |
| 2 | 692 | (0.69, 0.26) | 5 | 6.6 | 6.8 | 0.29 | 0.29 | 0.14 | 0.14 |
| 3 | 692 | (0.71, 0.27) | 4.6 | 6.4 | 6.6 | 0.27 | 0.28 | 0.14 | 0.14 |
| 4 | 684 | (0.68, 0.27) | 2.5 | 9.7 | 11 | 0.51 | 0.56 | 0.37 | 0.58 |
| 5 | 688 | (0.70, 0.27) | 2.5 | 8.4 | 9.2 | 0.41 | 0.46 | 0.3 | 0.48 |
| 6 | 688 | (0.72, 0.27) | 2.6 | 7.5 | 8.5 | 0.34 | 0.38 | 0.23 | 0.4 |
| 7 | 692 | (0.62, 0.23) | 5 | 5.0 | 5.9 | 0.21 | 0.3 | 0.1 | 0.14 |
| 8 | 700 | (0.66, 0.25) | 6 | 3.9 | 4.3 | 0.15 | 0.18 | 0.07 | 0.09 |
| 9 | 700 | (0.70, 0.26) | 6.5 | 3.3 | 3.6 | 0.12 | 0.13 | 0.05 | 0.06 |
| 10 | 692 | (0.62, 0.23) | 2.6 | 5.0 | 6 | 0.2 | 0.24 | 0.13 | 0.21 |
| 11 | 692 | (0.66, 0.25) | 2.6 | 5.1 | 6.1 | 0.18 | 0.2 | 0.12 | 0.19 |
| 12 | 700 | (0.70, 0.26) | 2.8 | 4.2 | 5.1 | 0.12 | 0.15 | 0.07 | 0.13 |
| 13 | 696 | (0.69, 0.28) | 3.3 | 5.3 | 6.4 | 0.27 | 0.29 | 0.16 | 0.27 |
| 14 | 700 | (0.70, 0.28) | 3.3 | 4.2 | 4.7 | 0.19 | 0.2 | 0.11 | 0.17 |
| 15 | 700 | (0.70, 0.28) | 3.3 | 3.2 | 3.3 | 0.13 | 0.14 | 0.08 | 0.11 |
| 16 | — | (0.70, 0.26) | — | 3.3 | — | 0.18 | — | 0.08 | — |
| 17 | — | (0.66, 0.25) | — | 3.6 | — | 0.21 | — | 0.12 | — |
| 18 | — | (0.58, 0.22) | — | 6.2 | — | 0.48 | — | 0.26 | — |
| 19 | — | (0.66, 0.25) | — | 6.4 | — | 0.41 | — | 0.21 | — |
| 20 | — | (0.70, 0.26) | — | 5.7 | — | 0.33 | — | 0.18 | — |
| 21 | — | (0.71, 0.26) | — | 4.9 | — | 0.26 | — | 0.14 | — |
| 22 | — | (0.71, 0.27) | — | 4.5 | — | 0.23 | — | 0.13 | — |
| 23 | — | (0.52, 0.21) | — | 5.2 | — | 0.41 | — | 0.3 | — |
| 24 | — | (0.61, 0.24) | — | 5.9 | — | 0.4 | — | 0.28 | — |
| 25 | — | (0.65, 0.25) | — | 6.7 | — | 0.4 | — | 0.26 | — |
| 26 | — | (0.68, 0.26) | — | 5.6 | — | 0.33 | — | 0.21 | — |
| 27 | — | (0.72, 0.27) | — | 0.01 | — | 0.001 | — | 0.02 | — |

As shown in Table 4, the organic luminescent units 1 to 27 emitted red or deep-red phosphoresecent lights.

Regarding to the unit 27, its organic luminescent layer only consisted of the compound of chemical formula (1) (100 wt %) and the wavelength of maximum emission was about 700 nm. However, its ηₑₓₜ at 100 cd/m² was merely 0.01%, which suggests the compounds of chemical formula (1) or (2) may need to integrate with other host materials to improve the luminescent efficiency of the unit.

Regarding to the units 1 to 3, in which the guest materials were the compound of chemical formula (1) with doping concentrations of 7 wt %, its ηₑₓₜ at 100 cd/m² was 6.8%. If the doping concentrations increased to 10 wt % and 15 wt %, the ηₑₓₜ were below 7% instead. The exciton may be effected by the triplet-triplet annihilation (TTA) effect caused by the increasing of the doping concentration of the guest materials, which results in the dropping of ηₑₓₜ. However, even for the unit 3 whose doping concentration of the guest material was as high as 15 wt %, the ηₑₓₜ was still as high as 6.4%. It may be because that the compound of chemical formula (1) has an excellent spiro structure which can effectively reduce the concentration-quenching effect. In addition, the turn-on voltages Vₒₙ of the units 1 to 3 were about 5V.

Regarding to the unit 4 to unit 6, the energy gap between the HOMO of the NPB host (−5.4 eV) and that of PEDOT is 0.5 eV, which is smaller than half of the energy gap between the CBP host and PEDOT. This difference and the Bebq₂ co-host both contributed to solve the problem caused by the high LUMO of NPB (−2.2 eV). Accordingly, the turn-on voltages Vₒₙ of the units 4 to 6 were about 2.5 V, which were 2.5 V lower than those of units 1 to 3 with CBP host. Such phenomenon may represent the electron injection barriers of the units 4 to 6 were actually reduced due to the configuration of the units 4 to 6. The triplet energy levels of the NPB host and Bebq₂ co-host were about 0.3 eV lower than that of the host CBP. However, the dopant compound had a comparatively lower triplet energy level, which was 1.81 eV. Under such circumstance, the exciton energy was not easy to return to the host, and it thus caused effective host-to-guest energy transfer. Therefore, the luminescent efficiencies were largely increased. In the unit 4 with a 7 wt % of chemical formula (1) as the guest material, it had a maximum EQE of 11% and ηₑₓₜ of 9.7% at 100 cd/m². It is 43% higher than that of the unit 1, which also has the same guest material of same concentration but with CBP host. In addition, when the doping concentration of the chemical formula (1) was increased to 15% in the unit 6, the ηₑₓₜ at 100 cd/m² was 7.5%, which was still higher than that (6.8%) of unit 3 having the same guest material of same concentration but has CBP host.

Regarding to the unit 7 to unit 9, in which the host materials were CBP and the guest materials were the compound of chemical formula (2) with various doping concentrations, their turn-on voltages Vₒₙ were also about 5 V, similar to those of the units 1 to 3. When the doping concentration of the guest material of chemical formula (2) was 7 wt %, the unit had a maximum EQE of 5.9% and its ηₑₓₜ at 100 cd/m² was 5.0%. When the doping concentration of the guest material of chemical formula (2) was increased to 15 wt %, the unit had a ηₑₓₜ at 100 cd/m² of 3.3%.

Regarding to the unit 10 to unit 12, in which various concentrations of the compound of chemical formula (2) as the guest materials blended in the co-host NPB/Bebq₂, their driving voltages Vₒₙ were 2.6 V, similar to those of units 4 to 6. Such phenomenon suggests that the electron injection barriers of the units 10 to 12 were actually reduced due to this configurations. When the doping concentration of the guest material of chemical formula (2) was 7 wt %, the unit had a maximum EQE of 6% and its ηₑₓₜ at 100 cd/m² was 5%. Comparing to the units 4 to 6 with similar unit structure, the units 10 to 12 have lower efficiencies, but have similar turn-on voltages. It may be attributable to that the compound of chemical formula (1) has a higher phosphorescence quantum yield than that of chemical formula (2).

Regarding to the unit 13 to unit 15, their host materials were WPH401 and their guest materials were the compound of chemical formula (2) with various doping concentrations. The LUMO energy level of the electron transport TAPC was about −2.0 eV, so that the electrons may be effectively restrained in the organic luminescent layer. Meanwhile, the WPH401 host has a lower LUMO energy level (about −2.7 eV) than NPB host does, and it has a better electron transfer efficiency with the electron transporter TPBI. Under such unit configuration, the energy may be effectively transferred from host to guest. When the doping concentration of the guest material of chemical formula (2) was 5 wt %, the unit 13 had a better performance. Its maximum EQE was 6.4%, which is about 7% higher than the unit 10 using the NPB/Bebq₂ co-host system did. The red color of the emission light of the unit 13 was deeper than that of the unit 10. Therefore, comparing to the compound of chemical formula (1), the iridium complex compound of chemical formula (2) was more feasible in a vacuum evaporation process, and the decomposition of the compound of chemical formula (1) occurred in the vacuum deposition process was therefore prevented. Also, the device performance of such units was very good and the device emits very deep red phosphorescence.

Regarding to the unit 23 to unit 26, in which the host materials were NPB and the guest materials were the compound of chemical formula (1) with doping concentrations of 7 wt % to 20 wt %, their ηₑₓₜ at 100 cd/m² were about 5.2% to 6.7%. However, comparing to the unit 27, a blue shift was occurred. It may be attributable to that the NPB host has a maximum emission wavelength of 444 nm. With increasing of the doping concentrations of chemical formula (1) from 7 wt % to 15 wt %, the degree of blue shift was gradually reduced and the EQEs were gradually increased. In addition, the units 23 to 26 had maximum luminance ranging from about 820 cd/m² to 1403 cd/m².

Regarding to the unit 18 to unit 22, in which the host materials were Spiro-2CBP and the guest materials were the compound of chemical formula (1) with doping concentrations of 5 wt % to 20 wt %, the red color of their emission lights were even deeper and their ηₑₓₜ at 100 cd/m² were about 4.5% to 6.4%. The unit 16, in which the host materials were SimCP2 and the guest materials were the compound of chemical formula (1) with doping concentrations of 15 wt %, had a ηₑₓₜ at 100 cd/m² of 3.3%; The unit 17, in which the host materials were TCTA and the guest materials were the compound of chemical formula (1) with doping concentrations of 15 wt %, had a ηₑₓₜ at 100 cd/m² of 3.6%.

From the data shown in Table 3, the unit 1 to unit 26 of the present experimental example not only had low driving voltages, but also had excellent ηₑₓₜ, η_{c}, and ηₚ. Accordingly, the guest materials provided by embodiments of the present disclosure had high electron-transfer efficiencies and hole-transfer efficiencies and the units can be driven by comparatively low voltage. In addition, the EQEs of the unit 1 to unit 26 were also high according to Table 3. Therefore, the guest materials provided by embodiments of the present disclosure have suitable triplet energy levels which help to reduce return of exciton energy and facilitate to increase the performance of the organic luminescent devices.

In summary, a series of red/deep-red phosphorescent guest materials are provided according to the present disclosure. The iridium complex compounds with cyclic quinoxaline-fused dibenzosuberene ligands, when they have structures represented by General Formula (1) and the atom A is General Formula (3), have excellent steric structure which may effectively reduce concentration-quenching effects. Meanwhile, such iridium complex compounds with cyclic quinoxaline-fused dibenzosuberene ligands are proven to be feasible in fabrication of an organic luminescent device through solution process. Suitable device configurations of units can also be found in the embodiments. For example, the unit with the structure of TO/PEODT:PSS/NPB:10% Bebq₂:5% Ir(QSTIF)₂(acac)/TPBI/LiF/Al has a maximum EQE of 11% and is a deep-red phosphorescent unit with high performance. When the iridium complex compounds have structures represented by General Formula (1) and the atom A is General Formula (2), they are proven to be feasible in fabrication of an organic luminescent device through vacuum deposition process and the decomposition of the materials is therefore prevented. The units having such guest materials have also good device performance and the device emits very deep-red phosphorescent light. For example, the unit with the structure of ITO/HATCN/TAPC/WPH401:5% Ir(DMQSTI)₂(acac)/TPBI/LiF/Al has a maximum EQE of 6.4%.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An iridium (III) complex compound with cyclic quinoxaline-fused dibenzosuberene ligand, comprising a structure of the following General Formula (1), 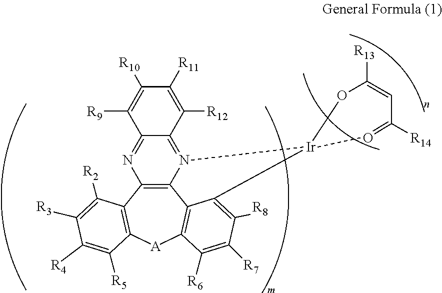
wherein m is 1 or 2, n is 1 or 2, and m+n is 3; A is a bridging atom represented by General Formula (2) or General Formula (3), 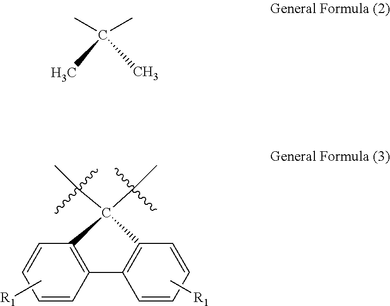
wherein R₁ is a hydrogen atom, alkyl or tert-butyl group, R₂ to R₁₄ are independently selected from the group consisting of hydrogen atom, halogen atom, cyano group, alkyl group, cycloalkyl group, aryl group, alkoxy group, amino group, haloalkyl group, thioalkyl group, silyl group and alkenyl group.

2. The iridium (III) complex compound of claim 1, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

3. The iridium (III) complex compound of claim 1, wherein m is 2 and n is 1.

4. The iridium (III) complex compound of claim 3, being represented by following chemical formula I or chemical formula II.

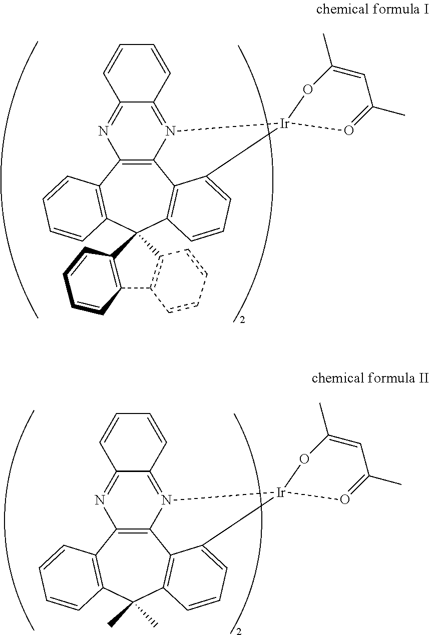

5. The iridium (III) complex compound of claim 3, having decomposition temperatures (T_{d}) ranged from 361° C. to 371° C.

6. The iridium (III) complex compound of claim 3, having oxidation potentials ranged from 0.55V to 0.60V and redox potentials ranged from −1.75V to −2.13V.

7. The iridium (III) complex compound of claim 3, having highest occupied molecular orbital energy level (E_{HOMO}) ranged from −5.35 eV to −5.4 eV and lowest unoccupied molecular orbital energy level (E_{LUMO}) ranged from −3.0 eV to −3.1 eV.

8. An organic electroluminescent device, comprising:
a first electrode layer;
a second electrode layer; and
an organic luminescent unit, deposited between the first electrode layer and the second electrode layer, wherein the organic luminescent unit has at least an iridium (III) complex compound with cyclic quinoxaline-fused dibenzosuberene ligand as shown in General Formula (1), 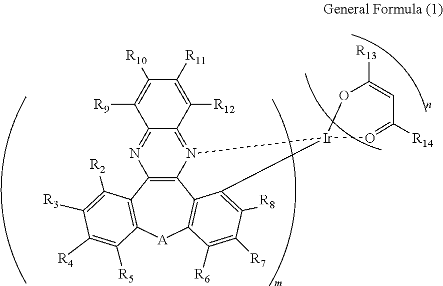
wherein m is 1 or 2, n is 1 or 2, and m+n is 3; A is a bridging atom represented by General Formula (2) or General Formula (3), 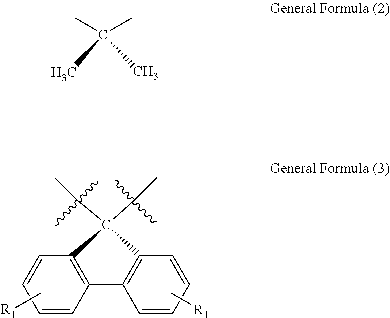
wherein R₁ is a hydrogen atom, alkyl or tert-butyl group, R₂ to R₁₄ are independently selected from the group consisting of hydrogen atom, halogen atom, cyano group, alkyl group, cycloalkyl group, aryl group, alkoxy group, amino group, haloalkyl group, thioalkyl group, silyl group and alkenyl group.

9. The organic electroluminescent device of claim 8, wherein the alkyl group is selected from the group consisting of a substituted or unsubstituted straight-chain alkyl group with the carbon number of 1 to 6, and a substituted or unsubstituted branched-chain alkyl group with the carbon number of 3 to 6, the cycloalkyl group is a substituted or unsubstituted cycloalkyl group with the carbon number of 3 to 6, the alkoxy group is selected from the group consisting of a substituted or unsubstituted straight-chain alkoxy group with the carbon number of 1 to 6, and a substituted or unsubstituted branched-chain alkoxy group with the carbon number of 3 to 6, the amino group is selected from the group consisting of secondary amino group and tertiary amino group, the haloalkyl group is selected from the group consisting of a substituted or unsubstituted straight-chain haloalkyl group with the carbon number of 1 to 6, and a substituted or unsubstituted branched-chain haloalkyl group with the carbon number of 3 to 6, the thioalkyl group is selected from the group consisting of a substituted or unsubstituted straight-chain thioalkyl group with the carbon number of 1 to 6, and a substituted or unsubstituted branched-chain thioalkyl group with the carbon number of 3 to 6, the silyl group is selected from the group consisting of a substituted or unsubstituted straight-chain silyl group with the carbon number of 1 to 6, and a substituted or unsubstituted branched-chain silyl group with the carbon number of 3 to 6, the alkenyl group is selected from the group consisting of a substituted or unsubstituted straight-chain alkenyl group with the carbon number of 2 to 6, and a substituted or unsubstituted branched-chain alkenyl group with the carbon number of 3 to 6.

10. The organic electroluminescent device of claim 8, wherein m is 2 and n is 1.

11. The organic electroluminescent device of claim 10, being represented by following chemical formula I and chemical formula II.

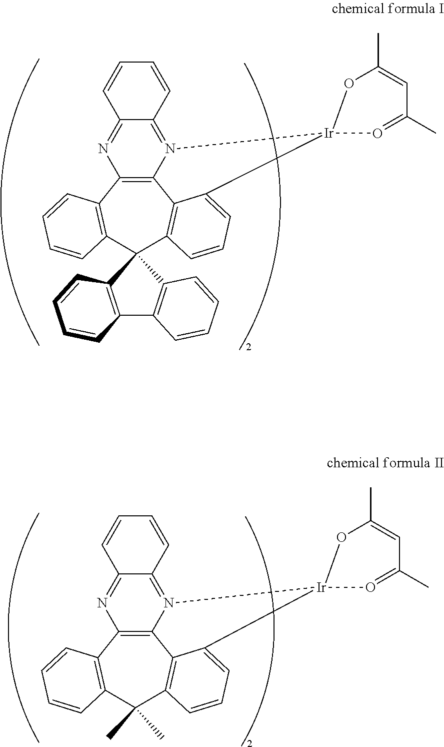

12. The organic electroluminescent device of claim 8, wherein the organic luminescent unit comprises an organic luminescent layer.

13. The organic electroluminescent device of claim 12, wherein the organic luminescent unit further comprises a hole transport layer and an electron transport layer, and the organic luminescent layer is deposited between the hole transport layer and the electron transport layer.

14. The organic electroluminescent device of claim 12, wherein the organic luminescent unit further comprises a hole injection layer, a hole transport layer, an electron transport layer and an electron injection layer, and the hole transport layer, the organic luminescent layer and the electron transport layer are sequentially deposited between the hole injection layer and the electron injection layer.

15. The organic electroluminescent device of claim 12, wherein the organic luminescent layer comprises a host material and a guest material, and the guests material comprises the iridium (III) complex compound with cyclic quinoxaline-fused dibenzosuberene ligand.

16. The organic electroluminescent device of claim 15, wherein the content of the guest material in the organic luminescent layer is between 3 wt % to 20 wt %.
